(19) United States
(12) Patent Application Publication
(10) Pub. No.: US 2008/0199857 A1
(43) Pub. Date: Aug. 21, 2008

(54) METHOD OF INCREASING SPECIFICITY OF NUCLEIC ACID HYBRIDIZATION USING ZWITTERIONIC COMPOUND

(75) Inventors: Jung-nam LEE, Yongin-si (KR); Sang-hyun PEAK, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/612,862

(22) Filed: Dec. 19, 2006

(30) Foreign Application Priority Data
Apr. 7, 2006 (KR) 10-2006-0031932

(51) Int. Cl. C12Q 1/68; C07H 1/00; C12P 19/34

(52) U.S. Cl. 435/6; 536/25.3; 435/91.2

(57) ABSTRACT

A method of increasing the specificity of nucleic acid hybridization, comprising hybridizing nucleic acid in a solution containing a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES) is provided. The method allows a reduction in the yield of non-specific amplification products in multiplex PCRs while maintaining the yield of target amplification products.

This application claims the priority to Korean Patent Application No. 10-2006-0031932, filed on Apr. 7, 2006, and all benefits accruing therefrom under 35 U.S.C. § 119, the disclosure of which is incorporated herein in its entirety by reference.

### BACKGROUND OF THE INVENTION

1. Field of the Invention

The present invention relates to a method of increasing the specificity of nucleic acid hybridization using specific zwitterionic compounds.

2. Description of the Related Art

“Hybridization specificity” (also known as “hybridization stringency”) refers to the percentage of nucleotides which must match on two unrelated single-stranded nucleic acid molecules before they will base pair with each other to form a duplex, given a certain set of physical and chemical conditions. In general, attempts have been made to control the degree of hybridization specificity by adjusting the salt concentration of the hybridization buffer, by adjusting the hybridization reaction temperature, by adding components such as Denhart's solution to the hybridization reaction, or by adding a non-specific DNA or RNA to the hybridization reaction to induce competition for hybridization. As used herein, the terms “hybridization specificity” and “specificity of nucleic acid hybridization” are used interchangeable.

Polymerase chain reaction (PCR) is a method of amplifying nucleic acids, comprising denaturation, annealing and extension of nucleic acids. During the process of annealing, hybridization of a primer nucleic acid and a target nucleic acid takes place. The conditions for the PCR can be altered according to the degree of homology between the primer nucleic acid and the target nucleic acid. For example, when the annealing temperature is raised in a given set of PCR conditions, the yield of non-specific hybridization between the primer and the target is decreased, whereas, when the annealing temperature is lowered, the yield of non-specific hybridization between the primer and the target is increased.

The hybridization specificity between the primer nucleic acid and the target nucleic acid exerts a significant effect on a polymerase chain reaction, and particularly on a multiplex PCR. Multiplex PCR is a variant of PCR, which enables simultaneous amplification of many target nucleic acids that are intended for amplification, in the same reaction, typically by using more than one pair of primers. More particularly, for a multiplex PCR reaction, different primer pairs, each of which is able to amplify a specific target nucleic acid sequence, are placed in one reactor in which amplification of the target nucleic acid sequences is performed simultaneously. Each primer pair used in the multiplex PCR should specifically hybridize to its target nucleic acid sequence and should not interfere with hybridization of other primer pairs with their respective target nucleic acids, so that each target nucleic acid can be sufficiently amplified in the same reaction.

A multiplex PCR results in multiple target amplification products. The multiplex PCR products can be analyzed by gel electrophoresis to yield a number of electrophoretic bands on the gel. If the hybridization specificity of the primer pairs is too low during the annealing process, amplification products resulting from non-specific hybridization may appear on the electrophoresis gel in addition to the bands of the targeted amplification products. If the hybridization specificity during the annealing process is adequate, only the specifically targeted bands will appear on the electrophoresis gel. Therefore, increasing the hybridization specificity is highly desirable.

U.S. Pat. No. 4,936,963 discloses a method to lower the overall conductivity in electrophoresis by using histidine, which is a zwitterionic substance, in order to reduce the time taken by electrophoresis.

In this regard, the inventors conducted research in order to determine and address the problems present in existing nucleic acid hybridization technologies. The inventors found that an increase in the specificity of nucleic acid hybridization can be induced by adding specific zwitterionic compounds to a nucleic acid hybridization reaction, especially a multiplex PCR.

### BRIEF SUMMARY OF THE INVENTION

In one embodiment, the invention is directed to a method of increasing the specificity of nucleic acid hybridization by hybridizing nucleic acids in a solution comprising a zwitterionic compound.

In another embodiment, the invention is directed to a method of increasing the specificity of nucleic acid hybridization, the method comprising hybridizing nucleic acids in a solution comprising a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES).

In another embodiment, the invention is directed to a method of amplifying a target nucleic acid, the method comprising hybridizing a primer and a target nucleic acid in a solution comprising a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES).

### BRIEF DESCRIPTION OF THE DRAWINGS

FIG. 1 is a photographic image of an electrophoretic gel showing the results of an electrophoretic analysis of PCR products obtained when 3-(cyclohexylamino)-1-propanesulfonic acid (CAPS) was added to a multiplex PCR according to the invention.

FIG. 2 is a spectrum obtained using an Agilent 2100 Bioanalyzer in a quantitative evaluation of the yields of target hybridization products and non-specific hybridization products.

FIG. 3 is a photographic image showing the results of an electrophoretic analysis of PCR products obtained when 3-(cyclohexylamino)-2-hydroxy-1-propane sulfonate (CAPSO) was added to a multiplex PCR according to the invention.

FIG. 4 is a photographic image showing the results of an electrophoretic analysis of PCR products obtained when 2-(cyclohexylamino)ethane sulfonate (CHES) was added to a multiplex PCR according to the invention.

FIG. 5 is a photographic image showing the results of an electrophoretic analysis of PCR products obtained when betaine was added to a multiplex PCR according to the invention.

### DETAILED DESCRIPTION OF THE INVENTION

The invention will now be described more fully with reference to the accompanying drawings, in which embodiments of the invention are shown. The invention may, however, be embodied in many different forms and should not be construed as being limited to the embodiments set forth herein, rather, these embodiments are provided so that this disclosure will be thorough and complete, and will fully convey the concept of the invention to those skilled in the art.

As used herein, the term “nucleic acid” means DNA or RNA. The DNA or RNA can be in any possible configuration, i.e. in the form of double-stranded (DS) nucleic acid, or in the form of single-stranded (ss) nucleic acid, or as a combination thereof (in part ds or ss).

Nucleic acid hybridization is a fundamental tool in molecular genetics, which takes advantage of the ability of individual single-stranded nucleic acid molecules to form double-stranded molecules, that is, to hybridize to each other. Typically, nucleic acid hybridization involves mixing single-stranded nucleic acid molecules from two sources of nucleic acids, such as, for example, mixing a probe, which typically consists of a homogeneous population of identified molecules, with a target nucleic acid molecule, which typically consists of a complex, heterogeneous population of nucleic acid molecules.

Hybridization of nucleic acids refers to the binding between nucleic acids of different origins. For hybridization to occur, the interacting single-stranded nucleic acid molecules must have sufficient sequence homology. The conditions for hybridization can be varied according to the sequence homology of the nucleic acids to be bound. Thus, if the sequence homology between the subject nucleic acids is high, stringent conditions are used. If the sequence homology is low, mild conditions are used. When the hybridization conditions are stringent, the hybridization specificity increases, and this increase of the hybridization specificity leads to a decrease in the yield of non-specific hybridization products. However, under mild hybridization conditions, the hybridization specificity decreases, and this decrease in the hybridization specificity leads to an increase in the yield of non-specific hybridization products. The presence of non-specific hybridization appears as false positive bands when a nucleic acid hybridization reaction is analyzed by gel electrophoresis. Accordingly, there is a need to reduce the level of the non-specific hybridization products. In order to reduce the level of non-specific hybridization products, an attempt was made to increase nucleic acid hybridization specificity by hybridizing nucleic acids in a solution containing a specific zwitterionic compound.

Hybridization between nucleic acids can occur between a DNA molecule and a DNA molecule, hybridization between a DNA molecule and a RNA molecule, and hybridization between a RNA molecule and a RNA molecule. Nucleic acid hybridization reactions include Southern hybridization reactions and Northern hybridization reactions. For the Southern hybridization reaction, a labelled DNA probe molecule hybridizes, or binds to a target DNA molecule. For the Northern hybridization reaction, a labelled a labelled DNA or RNA probe molecule hybridizes, or binds to a target RNA molecule.

Nucleic acid hybridization reactions are particularly important for applications of DNA chips, since a rapid detection of a target nucleic acid to a specific probe can be effected by the conditions the hybridization reaction is performed. In an exemplary DNA chip procedure, a probe is immobilized on a chip, and a sample containing the target nucleic acid is added to the immobilized probe, wherein the identity/abundance of the target nucleic acid is detected upon hybridization to the probe.

In one embodiment, the invention provides a method of increasing the specificity of nucleic acid hybridization by hybridizing nucleic acids in a solution containing a specific zwitterionic compound is provided. A zwitterionic compound refers to a compound that is electrically neutral but carries formal positive and negative charges, and which is capable of acting simultaneously as an acid as well as a base in a neutral solution. Examples of zwitterionic compounds include betaine, amino acids, 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), 2-(cyclohexylamino)ethane sulfonate (CHES), and the like. The zwitterionic compounds, betaine and CAPS, have structures as shown below:

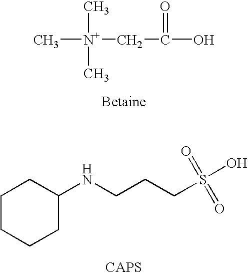

In most cases, zwitterionic compounds tend to increase the nucleic acid hybridization specificity in a nucleic acid hybridization reaction, thereby decreasing the yield of non-specific hybridization products. Unfortunately, the same zwitterionic compounds can also decrease the yield of target hybridization products. However, the addition of some zwitterionic compounds to the nucleic acid hybridization reaction show desirable characteristics in that they increase the nucleic acid hybridization specificity, thereby decreasing the yield of non-specific hybridization products, while maintaining the yield of target hybridization products. The inventors discovered zwitterionic compounds having such desirable characteristics, including 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), 2-(cyclohexylamino)ethane sulfonate (CHES), and the like.

In one embodiment, when such a zwitterionic compound is added to a nucleic acid hybridization reaction, the specificity of the nucleic acid hybridization reaction is increased. As a result, the generation of false positive bands in an electrophoretic gel analyzing a nucleic acid hybridization reaction can be reduced. The false positive bands are the results of non-specific nucleic acid hybridization, and are generated to a large extent when the specificity of a nucleic acid hybridization reaction decreases. These false positive bands make the separation of target hybridization products difficult, and adversely affect the process of separating target genes in terms of processing time and operating costs. Accordingly, elimination of such non-specific hybridization products from a nucleic acid hybridization reaction is strongly desired for the efficient separation of target nucleic acids, for example, target genes.

In one embodiment of the invention, the concentration of the zwitterionic compound may be about 0.2 to about 3% by weight. When the concentration of the zwitterionic compound is lower than about 0.2% by weight, the nucleic acid hybridization specificity is decreased, increasing the yield of non-specific hybridization products. When the concentration of the zwitterionic compound is higher than about 3% by weight, the yield of target hybridization products is also decreased.

In another embodiment, the invention provides a method of amplifying a target nucleic acid, comprising hybridizing a primer and a target nucleic acid in a solution containing a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES) is provided.

According to the current embodiment of the invention, the method of amplifying a target nucleic acid comprises denaturation of double stranded nucleic acids, annealing the primer and a target nucleic acid, and extension of the primer. Hybridization between a primer and a target nucleic acid occurs during the process of annealing. When the zwitterionic compound of the invention is added to the hybridization reaction, the specificity of the hybridization between the primer and the target nucleic acid is increased. This increase in the nucleic acid hybridization specificity leads to a decrease in the yield of non-specific amplification products, as only the desired target hybridization products are formed and specifically amplified.

Exemplary methods for DNA amplification include polymerase chain reaction (PCR), ligase chain reaction, stranded-displacement amplification, nucleic acid-based amplification, repair chain reaction, helicase chain reaction, QB replicase amplification, and ligation activated transcription. In an exemplary embodiment, amplification of a target nucleic acid can be performed using a polymerase chain reaction.

According to another embodiment of the invention, the PCR is a multiplex PCR. The method of amplifying a target nucleic acid according to the current embodiment is intended to increase the specificity of a nucleic acid hybridization reaction, and more specifically, is intended to eliminate non-specific hybridization products between primers and target nucleic acids in the multiplex PCR during the amplification of a target gene. Multiplex PCR is useful, for example, for the identification of a plurality of pathogens. In most cases zwitterionic compounds tend to increase the nucleic acid hybridization specificity in a nucleic acid hybridization reaction, thus decreasing the yield of non-specific products formed during the hybridization. Unfortunately, the same compounds can also decrease the yield of target hybridization products. However, the addition of particular zwitterionic compounds to the nucleic acid hybridization reaction show desirable characteristics in that they increase the nucleic acid hybridization specificity, thereby decreasing the yield of non-specific hybridization products, while maintaining the yield of target hybridization products. These desirable characteristics constitute an essential factor that enables accurate identification of specific pathogens in a multiplex PCR by decreasing non-specific amplification, i.e., false positive products in the multiplex PCR.

The production of non-specific hybridization products becomes a particular problem in a multiplex PCR. A multiplex PCR allows detection of a number of nucleic acids in a single PCR mixture. When analyzing the results of multiplex PCR, a plurality of bands representing the products of the multiplex PCR appear on an electrophoresis gel. If, for example, the multiplex PCR reaction is used to identify specific pathogens, the presence of multiple non-specific amplification products generated from non-specific hybridization of primers makes it difficult to accurately identify specific pathogens. Accordingly, it is necessary to reduce the number non-specific amplification products produced by non-specific hybridization of primers in the multiplex PCR, and thereby reduce the number of bands appearing on a gel analyzing the results of the multiplex PCR. When an appropriate amount of the zwitterionic compound according to an embodiment of the present invention is added to the multiplex PCR, the number of bands of non-specific amplification products appearing on a gel analyzing the results of the multiplex PCR is significantly decreased.

In another embodiment, the multiplex PCR eliminates non-specific amplification products, while retaining the yield of target amplification products. As discussed above, when an appropriate amount of the zwitterionic compound is added to the multiplex PCR, the number of bands of non-specific hybridization products appearing on a gel analyzing the results of the multiplex PCR is significantly decreased. However, if the yield of target hybridization products is also decreased along with the decrease in the non-specific hybridization products, the desired effect is not achieved. In most cases, zwitterionic compounds simultaneously decrease the yield of non-specific hybridization products and the yield of target hybridization products. However, when the zwitterionic compound according to the invention is used, a reduction in the yield of non-specific hybridization products and maintenance of the yield of target hybridization products are simultaneously, and favorably, achieved.

In another embodiment, the zwitterionic compound may be used in a concentration of about 0.2 to about 3% by weight. When the concentration of the zwitterionic compound is lower than about 0.2% by weight, the nucleic acid hybridization specificity is decreased, increasing the yield of non-specific hybridization products. When the concentration of the zwitterionic compound is higher than about 3% by weight, the yield of target hybridization products will be decreased.

According to another embodiment, the primer or probe is specific to the detection of a plurality of pathogens. When multiplex PCR is performed using the zwitterionic compound according to the invention, the number of bands of non-specific amplification products is decreased, while the yield of target amplification products is maintained. Thus, it is now possible to easily solve the problem of the prior art in which accurate detection of a plurality of nucleic acids is difficult due to the high yield of non-specific hybridization products. The detection of a plurality of nucleic acid sequences encompasses the analysis of gene sequences that are related to pathogens, genetic diseases and the like. For example, the most common viruses which cause respiratory diseases in human beings include measles virus, enterovirus, rhinovirus, SARS-associated coronavirus (SARS-coV), varicella zoster virus (VSV), adenovirus, human parainfluenza virus 1 (HPIV 1), human parainfluenza virus 2 (HPIV 2), human parainfluenza virus 3 (HPIV 3), influenza virus A (IVA), influenza virus B (IVB), respiratory syncytial virus A (RSVA), and respiratory syncytial virus B (RSVB). Rapid and specific detection of these viruses is essential to the diagnosis, prevention and treatment of respiratory diseases, and the viruses can be detected rapidly and accurately by performing multiplex PCR using the zwitterionic compound according to the present invention.

In another embodiment, the invention provides a solution for nucleic acid hybridization comprising a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES). The solution for nucleic acid hybridization according to the current embodiment contains various compounds needed in a nucleic acid hybridization reaction. When a nucleic acid hybridization reaction is performed using the solution for nucleic acid hybridization comprising the zwitterionic compound according invention in addition to the various compounds, the specificity of the nucleic acid hybridization is increased, and more specific hybridization results can be obtained. As noted above, nucleic acid hybridization reaction is particularly important for DNA chips. For a DNA chip reaction, a probe is immobilized on a chip, a sample containing a target nucleic acid is added thereto, and a nucleic acid hybridization reaction is performed using the solution for nucleic acid hybridization comprising a zwitterionic compound according to the invention, and thus the target nucleic acid which is specific to the probe can be rapidly detected.

According to another embodiment, the invention provides a nucleic acid hybridization kit comprising a solution for nucleic acid hybridization, the solution comprising a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES). The nucleic acid hybridization kit can comprise additional components that are necessary for nucleic acid hybridization, which is well known to those having ordinary skill in the art.

Hereinafter, the present invention will be described in further detail with reference to the following Examples. These Examples are for illustrative purposes only, and should not be construed to limit the scope of the present invention.

### EXAMPLE 1

### Effect of CAPS on the Specificity of Nucleic Acid Hybridization in a Multiplex PCR

For this example, genomic DNA (gDNA) was isolated from Haemophilus influenza, which is a main pathogen for respiratory infections, as a template for a multiplex PCR. 3-(cyclohexylamino)-1-propanesulfonic acid (CAPS) was used as the zwitterionic compound at a concentration of 0.5% or 1% to perform a multiplex PCR. The multiplex PCR was performed using a GeneAmp PCR system 9700 (ABI), with a PCR mixture (template gDNA 0.2 ng or 1 ng, CAPS 0.5% or 1%, 1×Taq Pol buffer, 200 μM each of dNTP mixture, 400 nM each of PCR primer (SEQ ID NO:1 through SEQ ID NO:10), and Taq Pol 5 units). The multiplex PCR started with an initial activation step at 95° C. for 1 minute to completely denature the DNA, followed by subjecting the PCR mixture to 25 PCR cycles (5 sec at 95° C., 13 seconds at 62° C., and 15 seconds at 72° C.), and followed by a final extension at 72° C. for 1 minute.

For Example 1, in order to quantitatively evaluate the extent of production of target amplification products and non-specific amplification products in the multiplex PCR, the PCR products were subjected to electrophoresis, and then detected by fluorescence using an Agilent 2100 Bioanalyzer.

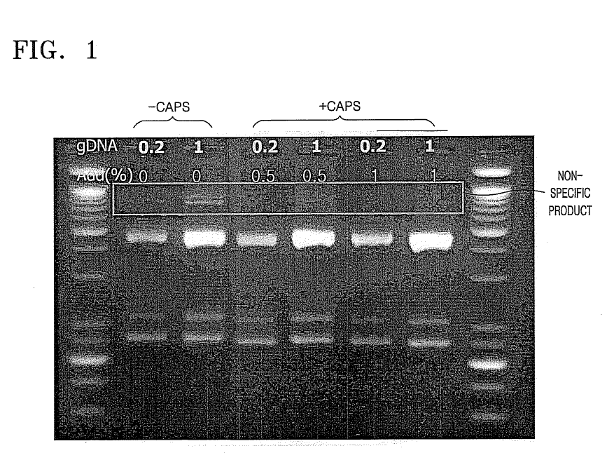

FIG. 1 is a photographic image of an electrophoresis gel showing the results of an electrophoretic analysis of PCR products obtained when CAPS was added to a multiplex PCR (Example 1). The results show a decrease in the yield of non-specific amplification products, indicating that non-specific hybridization was reduced. Referring to FIG. 1, it can be seen that for the samples having CAPS added to the multiplex PCR (indicated as +CAPS), the intensity of the bands of non-specific amplification products significantly decreased, as compared with the samples where the multiplex PCR was conducted without CAPS added (indicated as −CAPS). Further, FIG. 1 demonstrates that the intensity of non-specific amplification product bands markedly decreased as the concentration of CAPS increased. In the experiments having CAPS added (+CAPS) to the multiplex PCR, the intensity of the bands of non-specific amplification products markedly decreased, but the intensity of the target amplification product bands was maintained. Furthermore, as the initial amount of the template DNA increased, as anticipated, the amount of the target amplification products, and the amount of the non-specification amplification products increase.

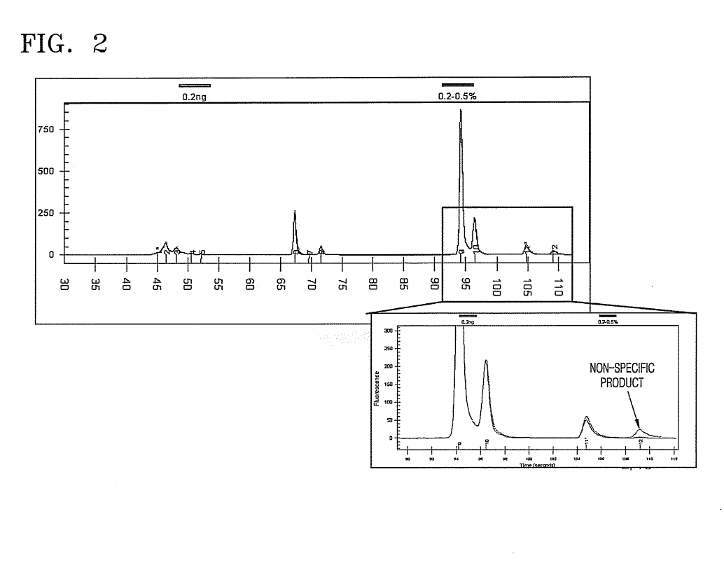

FIG. 2 is a scan of the gel obtained using an Agilent 2100 Bioanalyzer for quantitative evaluation of the yields of target amplification products and non-specific amplification products. Referring to FIG. 2, in the magnified portion of the graph below the main graph of FIG. 2, the upper curve of the two curves shown corresponds to the plot obtained from PCR using 0.2 ng of the gDNA but without CAPS, while the lower curve corresponds to the plot obtained from PCR using 0.2 ng of the gDNA in the presence of 0.5% CAPS. The band positions for the non-specific amplification products are indicated by the arrows. As shown in FIG. 2, the intensities of the target hybridization product bands were nearly consistent, regardless of whether CAPS was present in or absent from the multiplex PCR mixture. However, the bands of the non-specific amplification products were mostly eliminated when CAPS was present in the multiplex PCR mixture.

Therefore, it can be seen that when CAPS, a zwitterionic compound according the invention, is added to the multiplex PCR, the yield of non-specific amplification products is significantly decreased, while maintaining the yield of the target amplification products. Thus, addition of CAPS was found to be useful for increasing the specificity of primer hybridization with a target. Therefore, addition of CAPS to a hybridization solution is useful for detection of specific pathogens by hybridization of probes or primers specific for nucleic acid from the pathogens.

### EXAMPLE 2

### Effect of CAPSO and CHES on the Nucleic Acid Hybridization Specificity in Multiplex PCR

To investigate the effect that different types of the zwitterionic compounds had on the specificity of nucleic acid hybridization in multiplex PCR, the addition of 3-(cyclohexylamino)-2-hydroxy-1-propane sulfonate (CAPSO) and 2-(cyclohexylamino)ethane sulfonate (CHES) to multiplex PCR mixtures was examined. The same experiment described in Example 1 was performed, except that either CAPSO or CHES was added to the multiplex PCR at concentrations of 0.2%, 0.4%, 0.8% and 1.6%. Additionally, in Example 2, 1 ng of Haemophilus influenza gDNA template was used in the multiplex PCR.

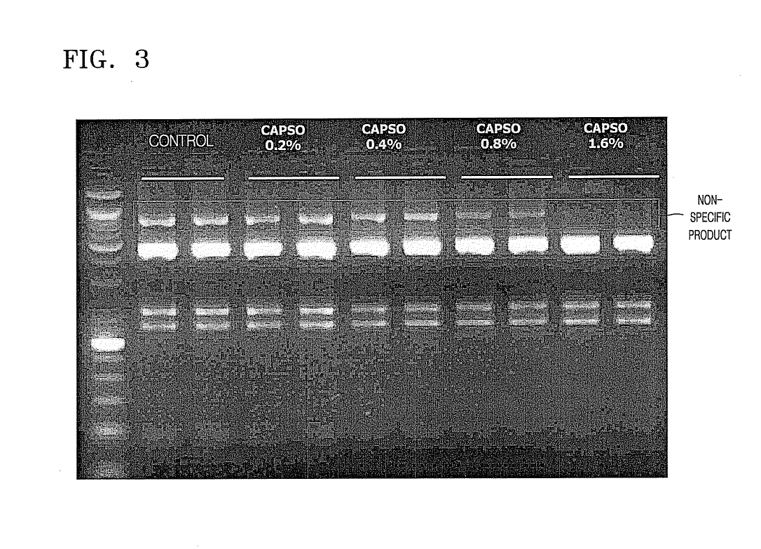

FIG. 3 is a photographic image of an electrophoresis gel showing the results of an electrophoretic analysis of PCR products obtained when CAPSO was added to a multiplex PCR. The control lanes represent a multiplex PCR with 0% CAPSO. Multiplex PCR at each CAPSO concentration was performed in duplicate. As can be seen in FIG. 3, the intensity of the band of the non-specific amplification products markedly decreased with increasing concentration of.

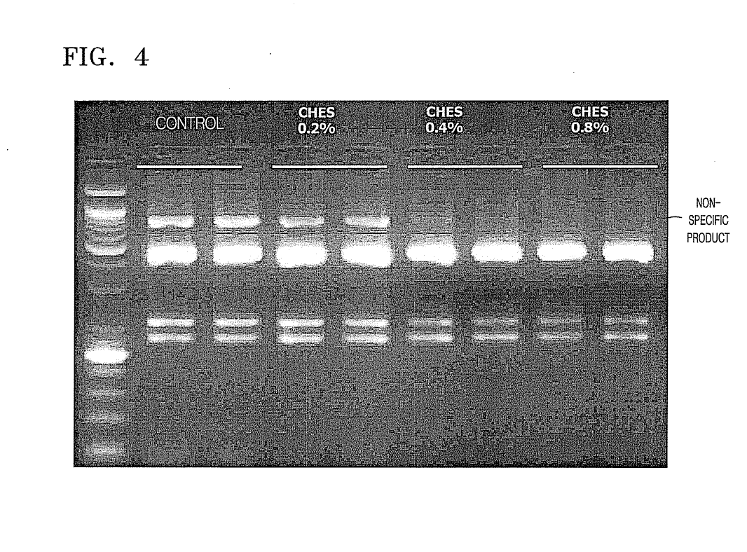

FIG. 4 is a photographic image of an electrophoresis gel showing the results of an electrophoretic analysis of PCR products obtained when CHES was added to a multiplex PCR. The results show a decrease in the yield of non-specific amplification products with increasing CHES concentration similar to the results with CAPSO shown in FIG. 3.

### EXAMPLE 3

### Effect of the Type of Zwitterionic Compound on Nucleic Acid Hybridization Specificity in a Multiplex PCR

For this example, betaine was used in investigating the specificity of nucleic acid hybridization in the presence of different types of zwitterionic compound in a multiplex PCR. The same experiment described in Example 1 was performed, except that betaine was added to a multiplex PCR using 5× betaine solution (Solgent, Inc.), in amounts of 2.5 μl (5% betaine), 5 μl (10% betaine), 10 μl (20% betaine), and 15 μl (30% betaine) and Haemophilus influenza gDNA template was used in the multiplex PCR in amounts of 1 ng, 0.1 ng and 0.01 ng, respectively.

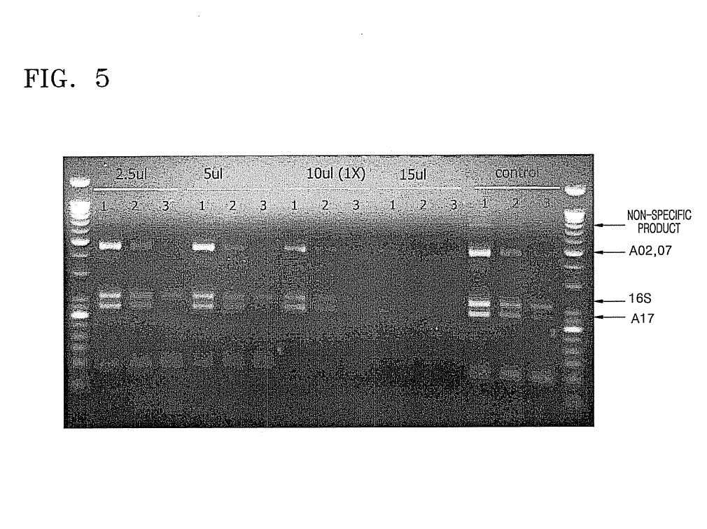

FIG. 5 is a photographic image of an electrophoresis gel showing the results of an electrophoretic analysis of PCR products obtained when betaine was added to a multiplex PCR. The results show a decrease in the yield of non-specific amplification products with addition of betaine to the multiplex PCR mixture. Referring to FIG. 5, the control was subjected to multiplex PCR without adding betaine. The numerals “1”, “2” and “3” labeling lanes at each betaine concentration refer to the amounts of the template gDNA used in the multiplex PCR, specifically 1 ng, 0.1 ng and 0.01 ng, respectively. As can be seen in FIG. 5, the intensity of the band of non-specific amplification products was markedly decreased when betaine was added to the reaction, as compared to the reactions in which betaine was not added. Further, higher concentrations of betaine resulted in a more noticeable decrease in the band intensity. However, it can be seen that unlike the case of CAPS, although the intensity of the non-specific amplification product band markedly decreased with an increasing concentration of betaine, the intensities of the bands of target amplification products also markedly decreased. Therefore, betaine is not shown to be suitable for the purpose of increasing the specificity of nucleic acid hybridization to detect specific pathogens.

Therefore, not all zwitterionic compounds show the effect of the present invention, and only specific zwitterionic compounds of the present invention, including CAPS, CHAPS, CAPSO, and CHES, can result in a decrease in the intensity of the band of non-specific amplification products, while maintaining the intensity of the band of target amplification products.

As discussed above, the method according to embodiments of the present invention allows reduction in the yield of non-specific amplification products while maintaining the yield of target amplification products in a multiplex PCR by increasing the specificity of nucleic acid hybridization (for example, primer-target hybridization). The method can be effectively used in the identification of specific pathogens, diagnosis of genetic diseases, analysis of gene sequences, and the like.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of increasing the specificity of nucleic acid hybridization, the method comprising
hybridizing nucleic acids in a solution comprising a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES).

2. The method of claim 1, wherein the concentration of the zwitterionic compound in the solution is about 0.2 to about 3% by weight.

3. The method of claim 1, wherein the hybridization is selected from the group consisting of hybridization between a DNA molecule and a DNA molecule, hybridization between a DNA molecule and a RNA molecule, and hybridization between a RNA molecule and a RNA molecule.

4. A method of amplifying a target nucleic acid, the method comprising
hybridizing a primer and a target nucleic acid in a solution comprising a zwitterionic compound selected from the group consisting of 3-(cyclohexylamino)-1-propanesulfonic acid (CAPS), 3-[(3-cholamidopropyl)dimethylammonio]-1-propane sulfonate (CHAPS), 3-(cyclohexylamino)-2-hydroxy-1-propane sulfonate (CAPSO), and 2-(cyclohexylamino)ethane sulfonate (CHES); and
amplifying the target nucleic acid.

5. The method of claim 4, wherein the amplification of the target nucleic acid is performed using a polymerase chain reaction (PCR).

6. The method of claim 5, wherein the polymerase chain reaction is a multiplex PCR.

7. The method of claim 4, wherein the primer is specific to the detection of a plurality of pathogens.

8. The method of claim 4, wherein the concentration of the zwitterionic compound is about 0.2 to about 3% by weight.


SEQUENCE LISTING (10 sequences)

SEQ ID NO: 1 - DNA, 23 bp, Artificial Sequence
  forward primer
gcgtaccttttgtataatgggtc

SEQ ID NO: 2 - DNA, 19 bp, Artificial Sequence
  reverse primer
gaccttagctggcggtctg

SEQ ID NO: 3 - DNA, 19 bp, Artificial Sequence
  forward primer
tgtcgggtaagttccgacc

SEQ ID NO: 4 - DNA, 19 bp, Artificial Sequence
  reverse primer
crgaaccaccggatcacta

SEQ ID NO: 5 - DNA, 20 bp, Artificial Sequence
  forward primer
cacctcgatgtcgrctcatc

SEQ ID NO: 6 - DNA, 21 bp, Artificial Sequence
  reverse primer
ggtcctctcgtactagrarca

SEQ ID NO: 7 - DNA, 22 bp, Artificial Sequence
  forward primer
tagcatatcagaaggcacaccc

SEQ ID NO: 8 - DNA, 23 bp, Artificial Sequence
  reverse primer
atccactcaagagagacaacatt

SEQ ID NO: 9 - DNA, 20 bp, Artificial Sequence
  forward primer
yccakactcctacgggaggc

SEQ ID NO: 10 - DNA, 22 bp, Artificial Sequence
  reverse primer
gtattaccgcrrctgctggcac